(19) United States
(12) Patent Application Publication
(10) Pub. No.: US 2006/0184001 A1
(43) Pub. Date: Aug. 17, 2006

(54) Method for MRI scanning of animals for transmissible spongiform encephalopathies

(76) Inventors: Lawrence A. Minkoff, Lattingtown, NY (US); Joel M. Stutman, Farmingdale, NY (US); Alexia L. McKnight, Chadds Ford, PA (US)

Correspondence Address: DARBY & DARBY P.C., P. O. BOX 5257, NEW YORK, NY, 10150-5257, US

(21) Appl. No.: 11/346,083

(22) Filed: Feb. 1, 2006

Related U.S. Application Data
(60) Provisional application No. 60/649,769, filed on Feb. 2, 2005.

(51) Int. Cl. A61B 5/05

(52) U.S. Cl. 600/410; 600/409

(57) ABSTRACT

A method for detecting the presence of various transmissible spongiform encephalopathy (TSE) in mammals is provided. Steps include taking a magnetic resonance imaging (MRI) image of the brain of the mammal, selecting a section within the image, determining a measurement of a first brain structure in the section, and determining a measurement of a second brain structure in the section. The ratio of the measurements is calculated and used to determine the probability that the mammal has a TSE. Areas and/or volumes of the first and second brain structures may be used. In one embodiment, the image section is sagittal or axial. An embodiment uses the lateral ventricle or frontal lobe as the first brain structure, and the cerebrum as the second brain structure. The method of determining the areas and volumes of the brain structures is provided. MRIs that can be used along with the MRI parameters are provided.

### CROSS-REFERENCE TO RELATED APPLICATIONS

Priority is claimed to U.S. Provisional Patent Application Ser. No. 60/649,769, filed on Feb. 2, 2005. The content of this priority application is incorporated into the present disclosure by reference and in its entirety.

### FIELD OF THE INVENTION

The present invention relates to a method of detection of transmissible spongiform encephalopathies in mammals using MRI. The present invention also relates to finding the ratio of measurements of a first brain structure to a second brain structure using MRI to determine the probability that a mammal has a transmissible spongiform encephalopathy. Also, the present invention relates to the use of a portable MRI modified to position a mammal to perform the above methods.

### BACKGROUND OF THE INVENTION

Transmissible spongiform encephalopathies (TSEs, also known as prion diseases) are devastating diseases which can afflict humans and non-human animals alike. TSEs are characterized by an indicative “spongy” morphology observed within the brain (Kübler et al. (2003) Br. Med. Bull. 66:267-279). Not only are these diseases devastating to human and animal health, they have wreaked havoc on livestock industries (such as those for cattle and sheep, see below). TSEs have been documented as transmissible from disparate mammalian species (see, for example, Hill and Collinge (2003) Br. Med. Bull. 66:161-170; Chesebro (2003) Br. Med. Bull. 66:1-20; Kübler et al. (2003) Br. Med. Bull. 66:267-279), and are invariably fatal after manifestation of clinical symptoms.

TSEs have extremely adverse affects on livestock industries. For example, within the United States and Canada, losses are projected to be in the billions of dollars due to the TSE bovine spongiform encephalopathy (BSE, also known as “Mad Cow Disease;” J. Am. Vet. Med. Assoc. News Apr. 15, 2004). Also, the sheep and goat industries of the U.S. are estimated to suffer losses of $20 million to $25 million per year due to the ovine TSE scrapie (Suszkiw (2002) Agricultural Research 50:14-16).

The United States Department of Agriculture (USDA) has developed surveillance programs and has reaction protocols should a particular TSE be discovered within the U.S. The oversight of these programs is part of the mission of the Animal and Plant Health Inspection Service (APHIS) of the USDA. APHIS has programs for BSE and scrapie as well as a program for chronic wasting disease (CWD), a TSE in wild and farmed deer and elk. APHIS also plays a major role in determining for which countries import restrictions will be imposed based on findings of TSEs in the livestock from those countries.

Problems exist, however, with the current U.S. programs. For example, a trade treaty between the United States and Mexico allows the importation of close to 250,000 head of sheep from Mexico. The flocks imported from Mexico are not tested, nor is there any assurance that every sheep in the flock is not infected with scrapie. This importation thwarts the attempts of the USDA to rid U.S. flocks of the disease.

Although many approved TSE tests are currently available, most are post-mortem (Kübler et al. (2003) Br. Med. Bull. 66:267-279) requiring the sacrifice of the animal for the sole purpose of testing. An invasive, pre-mortem “third eyelid” test for scrapie in sheep is currently used by the USDA, but it is not applicable in all scrapie infections (O'Rouke, et al. (2000) J. Clin. Microbiol. 38:3254-3259). For BSE, a live animal assay is thought to be far off, if attainable at all (Perkel (2004) The Scientist 5:20040112-02). Pre-symptomatic tests are not currently available for most TSEs (Soto (2004) Nat. Rev. Microbiol. 2:809-819) and pre-symptomatic diagnosis is reportedly virtually impossible (Aguzzi and Polymenidou (2004) Cell 116:313-327), except for scrapie (O'Rouke, et al. (2000) J. Clin. Microbiol. 38:3254-3259). Biochemical pre-mortem tests are being developed for other TSEs but they suffer from many disadvantages, for example, low sensitivity (Soto (2004) Nat. Rev. Microbiol. 2:809-819). The currently available tests take considerable time and are technically demanding (Kübler et al. (2003) Br. Med. Bull. 66:267-279).

Given the above, currently the only practical testing method for scrapie is the “third eyelid” test. This is an immunohistochemical test requiring the nictitating membrane (a.k.a. the “third eyelid”) to be removed from a sheep's eye. The third eyelid is correspondingly stored and shipped to an approved USDA testing facility. The only current USDA testing facility is in Ames, Iowa. The Ames facility does not have the resources to test even the 250,000 head of sheep from Mexico, let alone the millions of head of sheep on U.S. soil. In addition, the time required between taking a sample and receiving the results is lengthy (on the order of days to weeks); and the test is costly.

Therefore, there is a long-felt need to reliably detect TSE diseases in animal populations destined for import or for slaughter using a method that is fast, relatively inexpensive, non-invasive and may be performed on live animals, even those in pre-symptomatic stages of disease.

The present invention meets a long-felt need for a non-invasive, pre-mortem, easy, fast, and inexpensive test for TSEs through the use of novel methods using magnetic resonance imaging (MRI) technology.

### SUMMARY OF THE INVENTION

The present invention provides a method for detecting the presence of a transmissible spongiform encephalopathy (TSE) in a mammal including the steps of taking a magnetic resonance imaging (MRI) image of the brain of the mammal, selecting a section within the image, determining a measurement of a first brain structure in the section, and determining a measurement of a second brain structure in the section. Once the sections are measured, the ratio of the measurements of the first brain structure to the second brain structure is calculated and used to determine the probability that the mammal has a TSE. The present invention uses areas and/or volumes of the first and second brain structures. The method can be used on living mammals, whether alert, sedated, or anesthetized. The present method can be used with ovine, bovine, or cervid mammals.

In one embodiment, the image section is typically sagittal or axial sections of the brain. An embodiment uses the lateral ventricle or frontal lobe as the first brain structure, and the cerebrum as the second brain structure. In addition, the method of determining the areas and volumes of the brain structures is provided. Various TSEs may be detected using this method. Another embodiment includes the MRIs that can be used to perform the method of the present invention along with the MRI parameters.

### BRIEF DESCRIPTION OF THE FIGURES

The above and still further objects, features and advantages of the present invention will become apparent upon consideration of the following detailed description of a specific embodiment thereof, especially when taken in conjunction with the accompanying drawings wherein like reference numerals in the various figures are utilized to designate like components, and wherein:

FIG. 1A is a representative axial section of a healthy sheep brain;

FIG. 1B is a representative sagittal section of a healthy sheep brain. The section contains the largest contiguous region of the lateral ventricle. This section can be identified in that the lateral ventricle extends into the olfactory lobe;

FIG. 2A is an axial section of a healthy sheep brain;

FIG. 2B is an axial section of a scrapie infected sheep brain;

FIG. 2C is a sagittal section of a healthy sheep brain;

FIG. 2D is a sagittal section of a scrapie infected sheep brain;

FIG. 3 is a flow chart illustrating a method of the present invention;

FIG. 4A is a simplified histogram summarizing percent ventricle area of cerebrum area for control and scrapie-infected sheep;

FIG. 4B is a histogram of lateral ventricle area/(cerebrum area−lateral ventricle area) for control and scrapie-infected sheep;

FIG. 5 is a histogram summarizing percent of ventricle to cerebrum compared to the age of the sheep;

FIG. 6 is a simplified histogram summarizing percent ventricle area to frontal lobe area for control and scrapie-infected sheep;

FIG. 7 is a flow chart illustrating another method of the present invention;

FIG. 8 is a perspective view of a MRI device of the present invention;

FIG. 9 is a perspective view of another embodiment of the MRI device of the present invention;

FIGS. 10A and 10B are perspective views of a portable MRI device of the present invention;

FIG. 11 is a perspective view of a handheld MRI device of the present invention;

FIG. 12 is a detailed histogram (of FIG. 4A) summarizing percent ventricle area to cerebrum area for control and scrapie-infected sheep providing the break-down of the various test results;

FIG. 13 is a detailed histogram (of FIG. 6) summarizing percent ventricle area to frontal lobe area for control and scrapie-infected sheep providing the break-down of the various test results; and

FIG. 14 is a second detailed histogram summarizing percent ventricle area to cerebrum area for control and scrapie-infected sheep providing the break-down of the various test results.

### DETAILED DESCRIPTION

Early detection of transmissible spongiform encephalopathy (TSE, also known as prion disease) is important for the economics of livestock breeding and to avoid a potential threat to human health. There is an unmet need in the art for a reliable method of detection of TSEs in live animals. The invention described herein uses MRI to reliably detect TSEs in live animals, thus providing a method to reliably cull diseased animals for a herd to prevent horizontal transmission and keep diseased animals out of the food chain.

TSEs are recognized to exist in many animal species, including humans. Sheep and goats may be afflicted with scrapie, cattle with bovine spongiform encephalopathy (BSE), mink with transmissible mink encephalopathy (TME), and deer and elk with chronic wasting disease (CWD). Within humans, the various TSEs include sporadic, variant, and familial Creutzfeldt-Jakob disease (sCJD, vCJD, and fCJD, respectively); Gerstmann-Straussler-Sheinker syndrome (GSS); fatal familial insomnia (FFI); and kuru. The clinical symptoms associated with the separate TSEs are varied. However, there are commonalities, including behavioral changes,and locomotor incoordination (and ultimately death), although these symptoms may not all manifest within every afflicted animal.

Transmission of TSEs from one species to another has been observed (see, for example, Hill and Collinge (2003) Br. Med. Bull. 66:161-170; Chesebro (2003) Br. Med. Bull. 66:1-20; Kübler et al. (2003) Br. Med. Bull. 66:267-279). However, a species barrier to transmission is present that directly correlates to the length of incubation prior to the onset of symptoms. The species barrier depends on polymorphisms in the prion protein, strain type, route of inoculation, and dose of exposure (Chesebro (2003) Br. Med. Bull. 66:1-20; Hill and Collinge (2003) Br. Med. Bull. 66:161-170). The barrier is less pronounced for transmission from one animal to another within the same or related species, whereas the barrier is much higher for transmission between more disparate species. A higher barrier corresponds to a longer incubation period prior to the onset of clinical symptoms. However, the higher initial barrier decreases following passage of the disease within the same species (Hill and Collinge (2003) Br. Med. Bull. 66:161-170).

Animals afflicted with a TSE may or may not develop clinical symptoms (Collins et al. (2004) Lancet 363:51-61; Hill and Collinge (2003) Br. Med. Bull. 66:161-170). If an animal exhibits symptoms and subsequently dies of a TSE, the period prior to the observable symptoms is a pre-clinical stage of the disease. However, some animals are afflicted with a TSE but live a normal life span. In this case, the disease is sub-clinical in that it does not manifest and apparently does not cause death. It is difficult to determine by extrapolation, had the animal lived longer, if the disease would have manifested and ultimately caused death. In either case, (i.e., pre-clinical or sub-clinical disease), the disease is still transmissible from the infected animal to other animals. Therefore, a technique to determine whether an animal has a TSE prior to the onset of clinical symptoms is highly beneficial to halt the spread of the disease.

The transmission of TSEs may occur horizontally or vertically (Kübler et al. (2003) Br. Med. Bull. 66:267-279). Horizontal transmission occurs from infected animals to other animals not through the germ line and not from a parent to offspring. Vertical transmission occurs from parent to offspring. According to APHIS, in sheep, for example, a mechanism of the spread of scrapie is thought to occur through contact with the placenta and placental fluids by the newly born offspring (vertical) and other lambs on the pasture (horizontal). Horizontal transmission of scrapie to scrapie-free sheep re-introduced to pastures once grazed by scrapie-infected sheep has been observed more than three years after the eradication of scrapie-inflected flocks (Chesebro (2003) Br. Med. Bull. 66:1-20).

The hallmark of almost all TSEs is the accumulation of an abnormal isoform of the prion protein PrP. Pathological abnormalities resulting from accumulation of PrP are predominantly found within the central nervous system (CNS), although the lymphatic system may also show pathology (see, for example, Aguzzi and Polymenidou (2004) Cell 116:313-327; O'Rouke, et al. (2000) J. Clin. Microbiol. 38:3254-3259). Fatal familial insomnia (FFI) does not show large amounts of PrP (Hill and Collinge (2003) Br. Med. Bull. 66:161-170). However, the disease is transmissible, exhibits severe TSE clinical symptoms, and presents neurological pathology (see, for example, Tabernero et al. (2000) J. Neurol. Neurosurg. Psychiatry 68:774-777).

PrP is encoded by the PRNP gene on chromosome 20 and most highly expressed in neurons (Collins et al. (2004) Lancet 363:51-61 and references therein). The function of native PrP is not known but has been implicated in diverse activities, including circadian rhythm regulation, synaptic transmission, nerve fiber organization, copper ion trafficking, nucleic-acid chaperoning, anti-oxidant processes, and anti-apoptotic processes (Kovács et al. (2004) World J. Biol. Psychiatry 5:83-91; Collins et al. (2004) Lancet 363:51-61 and references therein).

PrP is known to exist in one of at least two isoforms (see, for example, Aguzzi and Polymenidou (2004) Cell 116:313-327; Collins et al. (2004) Lancet 363:51-61; Soto (2004) Nat. Rev. Microbiol. 2:809-819; and references therein). The normal protein, denoted PrP^{c} or PrP^{sen}, is α-helical in structure, globular, and susceptible (sensitive) to proteinase-K (PK) treatment. PrP^{c} is directed to the endoplasmic reticulum (ER) by a signal peptide. Within the ER, the protein is glycosylated and further modified by the addition of a glycosylphosphatidylinositol (GPI) anchor. PrP^{c} is ultimately directed to the cell surface where it associates with detergent-resistant microdomains (DRMs). The abnormal, misfolded protein, denoted PrP^{res} or PrP^{Sc}, has a greater percentage of β-sheet structure, self-aggregates, forms fibrils, and is mostly resistant to PK treatment. The PK resistant portion is termed PrP²⁷⁻³⁰. PrP^{res} is required for both prion replication and neuronal damage in TSEs. Genetic polymorphisms of the PrP protein exist with some more influential at causing disease than others. Since the PrP protein is naturally expressed, and thus is a “self” protein, there is no immune response against PrP^{res}.

There are currently two models of propagation of PrP^{Sc} (see, for example, Aguzzi and Polymenidou (2004) Cell 116:313-327; Soto (2004) Nat. Rev. Microbiol. 2:809-819). The first is the template-directed model. In this model, a normal PrP protein interacts with a misfolded PrP^{res} protein; and the interaction converts PrP^{c} into PrP^{res}. This interaction may be mediated by an unknown factor, denoted Protein X. The newly misfolded PrP protein may then either aggregate into fibrils or enter the propagation cycle again. Thus, in this model, the infectious agent is monomeric PrP^{res}. The second model of propagation is the seeded nucleation or nucleation-polymerization model. The normal and abnormal isoforms of the PrP protein are believed to exist in equilibrium, albeit the equilibrium greatly favoring the normal isoform. PrP protein molecules of the abnormal isoform may then interact with seed aggregates (such as those ingested from infected animals) or larger fibrils, which may break apart to also form seed aggregates. The seed aggregates of this model would then be the infectious agent further propagating the disease. One major difference between the two models is that, in the template-directed model, the conversion is through interaction with PrP^{res} instead of spontaneous as in the seeded nucleation model. Another major difference is that a small amount of PrP^{res} is always present in the seeded nucleation model, and thus the infectious agent could not be monomeric PrP^{res} as is the case for the template-assisted model. Otherwise, in the seeded nucleation model, all animals with PrP would develop TSEs since monomeric PrP^{res} is ubiquitous in this model.

Currently, there are several tests available to detect TSEs (see, for example, Perkel (2004) The Scientist 5:20040112-02; Kübler et al. (2003) Br. Med. Bull. 66:267-279; Soto (2004) Nat. Rev. Microbiol. 2:809-819). These tests detect the presence of PrP^{res} and thus require biological tissue. Presently, the relatively large amounts of PrP required for detection necessitates that the most affected areas, i.e. usually areas in the brain, be used for such tests. Therefore, the animals suspected of TSE infection must be sacrificed in order to perform most of these tests. Currently, efforts are underway to increase sensitivity; but these are being developed and still require invasive methods (Soto (2004) Nat. Rev. Microbiol. 2:809-819).

The tests available include a mouse bioassay. This test is considered the most sensitive yet is rather lengthy (Perkel (2004) The Scientist 5:20040112-02; Kübler et al. (2003) Br. Med. Bull. 66:267-279). Results are obtained on the timescale of months to one year. Infected tissue from a sacrificed animal is injected into the brains of mice, and the observation of TSE clinical signs in the inoculated mice is considered a positive result.

Immunohistochemistry (IHC) is considered the gold-standard of testing (Perkel (2004) The Scientist 5:20040112-02; Kübler et al. (2003) Br. Med. Bull. 66:267-279). However, this test “is still lengthier and more tedious than most common diagnostic tests” (Perkel (2004) The Scientist 5:20040112-02). Tissue samples from deceased animals exhibiting a spongiform morphology are stained with antibodies to PrP^{Sc} or to the astrocytic marker protein, glial fibrillary acidic protein (GFAP), to ascertain the extent of astrocytic gliosis. The stain is then visualized under a light microscope.

Other tests include those based on enzyme-linked immunosorbent assay (ELISA) techniques, conformational-dependent antibodies against the PrP^{Sc} isoform, or Western blots (see, for example, Soto (2004) Nat. Rev. Microbiol. 2:809-819; Aguzzi and Polymenidou (2004) Cell 116:313-327). Furthermore, more complicated tests include those that use spectroscopic techniques such as multispectral ultraviolet fluoroscopy (MUFS), confocal dual-color fluorescence correlation spectroscopy (FCS), Fourier-transform infrared spectroscopy (FTIR), or fluorescence detection after capillary electrophoresis (Soto (2004) Nat. Rev. Microbiol. 2:809-819, Kübler et al. (2003) Br. Med. Bull. 66:267-279). However, these are expensive techniques that are not readily amenable to high-throughput methods.

A pre-symptomatic, pre-mortem test has been performed to detect scrapie in sheep (O'Rouke, et al. (2000) J. Clin. Microbiol. 38:3254-3259). The test is an IHC test in that it uses a monoclonal antibody to stain lymphoid tissue from the nictitating membrane (third eyelid) of sheep. However, this test is invasive and takes a considerable amount of time since it is an IHC test. Also, a subset of sheep in the study “showed accumulation of PrP^{Sc} or a transmissible agent in CNS tissue but not lymphoid tissue” (O'Rouke, et al. (2000) J. Clin. Microbiol. 38:3254-3259). Therefore, the test is not applicable in all cases of scrapie infection.

Many questions persist in the prion science field (Aguzzi and Polymenidou (2004) Cell 116:313-327). There is still controversy as to what comprises (i) the infectious agent, (ii) the transmissible agent, and (iii) the neurotoxic agent (Chesebro (2003) Br. Med. Bull. 66:1-20; Kovács et al. (2004) World J. Biol. Psychiatry 5:83-91; Aguzzi and Polymenidou (2004) Cell 116:313-327). For instance, the viral theory of prion disease has not been disproved. However, since the hallmark of TSEs is the presence of the abnormal isoform of PrP, detection of TSEs through detection of PrP^{res} or the effects of this protein on tissues (even when PrP^{res} is undetectable by the test methods above) is independent upon the status of any such theories.

Magnetic resonance imaging (MRI) has been used to diagnose the Creutzfeldt-Jakob disease (CJD) transmissible spongiform encephalopathy in humans (Kovács et al. (2004) World J. Biol. Psychiatry 5:83-91; Mendez, et al. (2003) Neuroimaging 13:147-151; Collie et al. (2001) Clinical Radiology 56:726-739; Schröter et al. (2000) Arch Neurol. 57:1751-1757) and scrapie in infected mice (Sadowski et al. (2003) Neurosci. Lett. 345:1-4) and hamsters (Chung et al. (1995) Neurodegeneration 4:203-207; Chung et al. (1999) Neuroreport 10:3471-3477; Sadowski et al. (2003) Neurosci. Lett. 345:1-4). However, the protocols developed require the careful qualitative analysis of the generated MRI images by expert radiologists. Therefore, the detection of TSEs by these MRI protocols can be time intensive and vulnerable to individual interpretations of the images.

The present invention relates to the use of MRI to detect the presence of TSEs in mammals. MRI has revolutionized the practice of human and veterinary medicine. The technique is non-invasive; and the production of images is relatively fast, on the order of only a few minutes. MRI, and thus the present invention, can be performed on living subjects. The living subjects can either be conscious, sedated, or unconscious. The sedation of sheep has no effect on the MRI results of using the methods of the present invention.

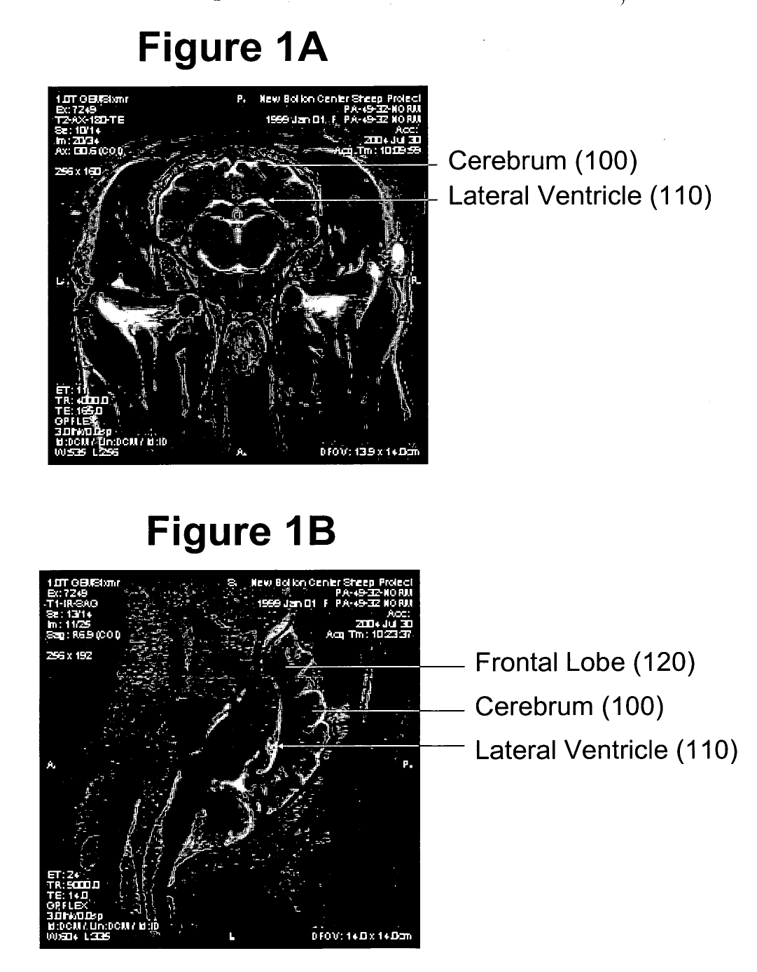

The images collected by MRI can be varied based upon the needs of the MRI operator. An image may be a cross-sectional section (slice) of a particular part of the body (e.g. the brain). Embodiments include brain image sections that may be axial (FIG. 1A), one that separates the brain into front and back parts, or sagittal (FIG. 1B), one that separates the brain into left and right parts. Alternatively, the brain section can be of any particular plane of interest. Furthermore, in other embodiments, the section thickness may be varied. Section thickness refers to the resolution of the image in the plane perpendicular to the image. One embodiment uses sections of 3 millimeter thickness.

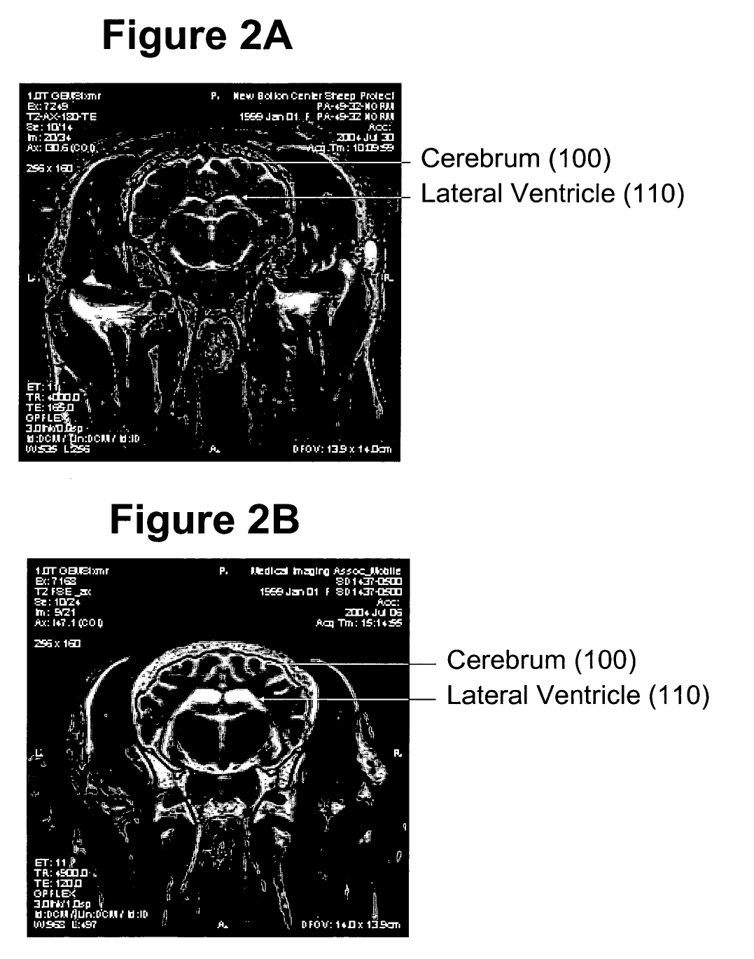

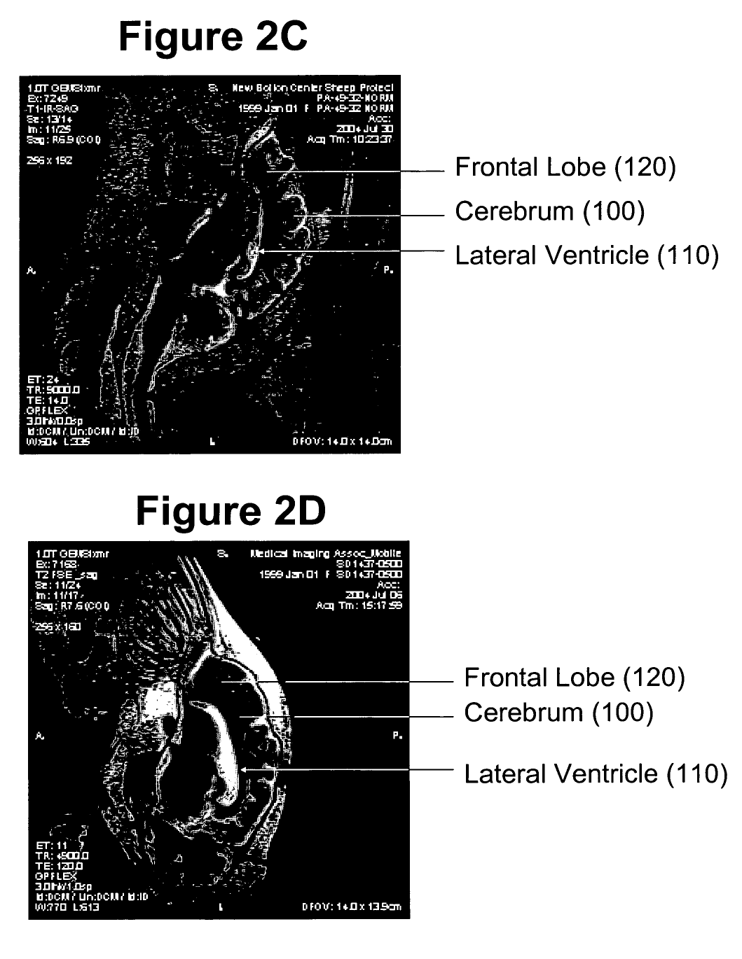

The accumulation of PrP^{res} can cause pathological changes in the morphology of the structures of the brain. These changes can include intracytoplasmic spongiosis, gliosis, and neuronal loss. The neuronal loss leads to brain atrophy which can be seen with MRI. (See FIGS. 2A, 2B, 2C, and 2D). However, the present invention is independent of the detection of PrP. The present invention uses the effects of a TSE, i.e. changes in structure, regardless if it is due to PrP. The present invention uses the fact that one structure of the brain atrophies (for example, the cerebrum) while another structure expands in response (for example, the lateral ventricle).

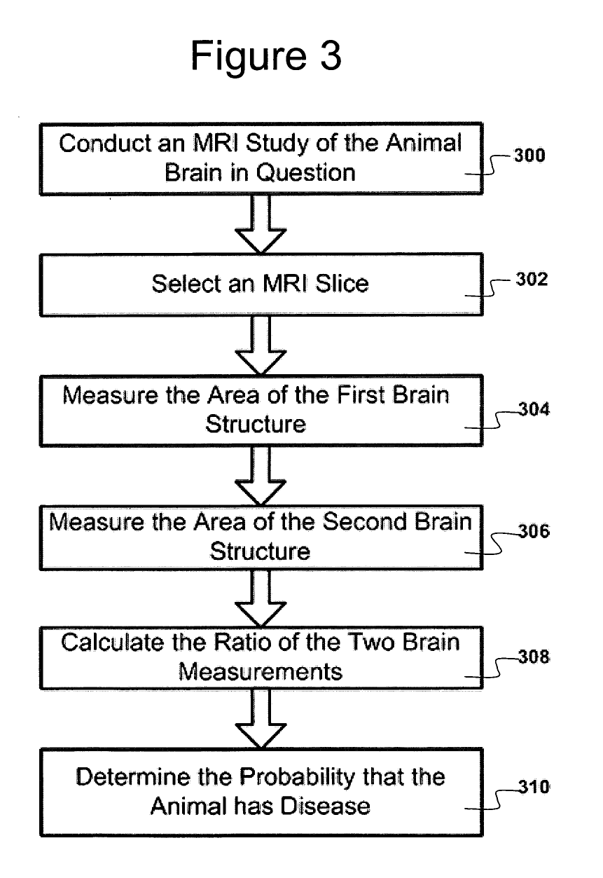

FIG. 3 illustrates a method of the present invention to detect the presence of a transmissible spongiform encephalopathy in a mammal. An MRI image of the brain of the mammal is taken (step 300). The images can then be processed manually, i.e. by an individual performing the following steps, or processed through automation, i.e. by a computer running one or more programs to perform the following steps. A section (slice) from the image is selected (step 302), and pertinent structures of the brain are delineated in the section. Alternatively, just one slice may be initially taken as the section selected instead of generating an image of the entire brain. The section may be sagittal, axial, or any other section determined by the MRI operator. The brain structures may include the lateral ventricle, the frontal lobe, the cerebrum, or any other brain structure found to be useful in the detection of TSEs by the methods of the present invention.

Measurements of a first brain structure within the selected section (slice) are calculated (step 304). In one embodiment, a first brain structure that is particularly useful is a lateral ventricle 110 (FIGS. 1A and 1B), although other structures may be used. Measurements of a second brain structure within the selected section (slice) are also determined (step 306). An embodiment uses the cerebrum 100 as the second brain structure (FIGS. 1A and 1B), but other structures may be used. In one embodiment, the measurements taken are the area of the two brain structures. The area of the first brain structure within the section can be determined by image manipulation computer software that can generate and measure the areas and/or volume of a multi-point polygonal boundary. An example of which is Adobe® Photoshop®. Adobe® Photoshop® allows areas of image files to be determined through features such as the Magnetic Lasso Tool. Any other computer software program that essentially performs the same function as the Lasso Tool of Adobe® Photoshop® also may be used.

Alternatively, any other computer software program may be used that is written to calculate the area of a structure within an image, especially when it comprises part of a larger computer software program designed for complete automation of the methods of the present invention. Automation of the methods of the present invention can reduce the uncertainty due to individual interpretations of the data by providing standardization. Other embodiments analyze the pixel density inside a particular section. Further, the software can analyze the pixel boundaries between the first and second brain structures to determine the separate brain structures and their measurements.

Other embodiments include using computer software that determines areas of regions by using bounding polygons. The software determines the boundaries of the polygon and then determines the area within the polygon. Many software programs are currently available to determine area using bounding polygons.

A template bounding polygon may be devised such that a standard polygon is used. A polygon of many points (for example, 300-500 points) may be determined to encompass a specific feature of an MRI image by a programmer using a standard MRI image. The standard image may be a healthy animal MRI scan. Through the use of statistics, a best fit may be determined using the template polygon superimposed upon another MRI scan. A high-pass filter over the image may be run to properly line up the polygon. This procedure may be completely automated.

The exact measurement, here exemplified as an area, can be calculated. The field of view and number of pixels within an MRI image may be adjusted through the MRI settings by the MRI operator. Through these settings, the actual dimensions represented by each pixel may be determined, and the measurement of the structure, an area in this embodiment, may be calculated.

The ratio of the two areas is then calculated (step 308). For example, the area of the first brain structure is divided by the area of the second brain structure. This ratio is then used to determine the probability that the mammal has a TSE (step 310). The ratio may be represented as a percent value.

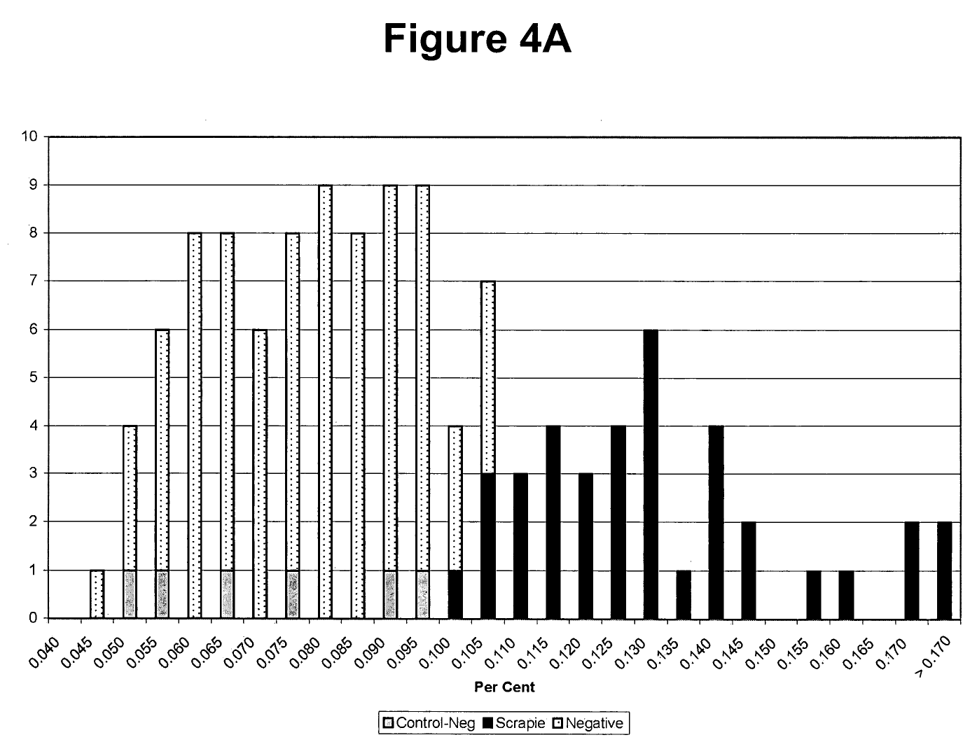

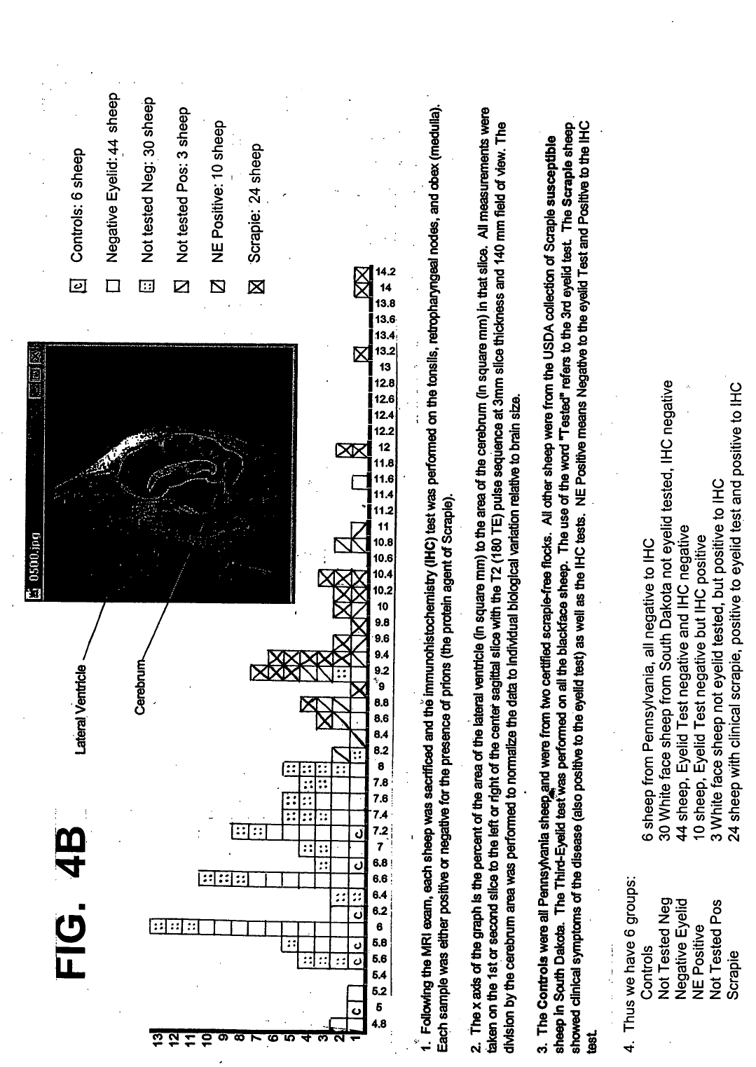

The determination of the probability that a mammal has a TSE is made based on the ratio value. The probability is empirically determined based on a large number of observations of tested mammals. The data from these observations may be represented as a histogram (See FIGS. 4-6, 12, and 13) where the number of animals tested is plotted against the ratio determined (in the figures represented as percent values). The probability determined from the ratio is dependent upon the particular first and second brain structures selected.

The calculations comparing the first and second brain structures simplify the detection of a TSE in the brain. Visually analyzing the image for signs of the disease is almost impossible. Turning to FIGS. 1A and 1B; 2A and 2B; and 2C and 2D, healthy and scrapie infected forms are difficult to distinguish by eye alone. The only exception is that the size of the lateral ventricle is larger in the scrapie infected brain

Another method of the present invention calculates brain structure volumes. Multiple contiguous sections (slices) of the MRI image of the brain may be used to reconstruct the brain structures in three-dimensions. The sections may be sagittal, axial, or any other determined to be useful in practicing the methods of the present invention. MRI images are taken (step 700) and multiple contiguous sections through a brain structure within an MRI image are selected (step 702). Areas of each of these sections are determined by any of the methods as described above (step 704). Each area is then multiplied by the thickness of each section to give the volume of the structure within each section (step 706). All of the contiguous sections containing the brain structure are then added together to give the volume of the brain section (step 708). This procedure is performed for both the first and second brain structures.

As a specific example, a computer program can take a multisection (multislice) MRI scan of an entity, the brain, for example, containing two or more regions of interest, e.g. two brain structures. A nested loop is then performed. For each section (slice) in the scan, each of the regions of interest are measured. For example, the area of one particular region of interest is measured. The area is then multiplied by the section (slice) thickness and added to a running total for the particular region of interest. The next region of interest is measured and has its multiplied area added to its running total. This is performed for every slice within the MRI scan. When the program is completed, the total volume for each region of interest is calculated.

The ratio of the first to second brain structures is determined by dividing the volume of the first brain structure by the volume of the second brain structure (step 710). This ratio, which may be represented as a percent value, is then used to determine the probability that the scanned mammal has a TSE (step 712). The probabilities are determined using the volumes of the first and second brain structures using another set of data generated from a large number of observations of tested mammals.

The ratio values that give particular probabilities that a mammal has a TSE are dependent upon the methodology used. Different brain structures may be used, and different types of measurements may be used (area, volume, etc.), for example. Therefore, the particular ratio value that may be used as a cut-off value corresponding to a high probability that the mammal has a TSE may change. However, regardless of the methodology, there is a cut-off value that may be selected within each methodology that will give zero false negatives. The present invention relies on the observation that one brain structure decreases in size and another expands to compensate.

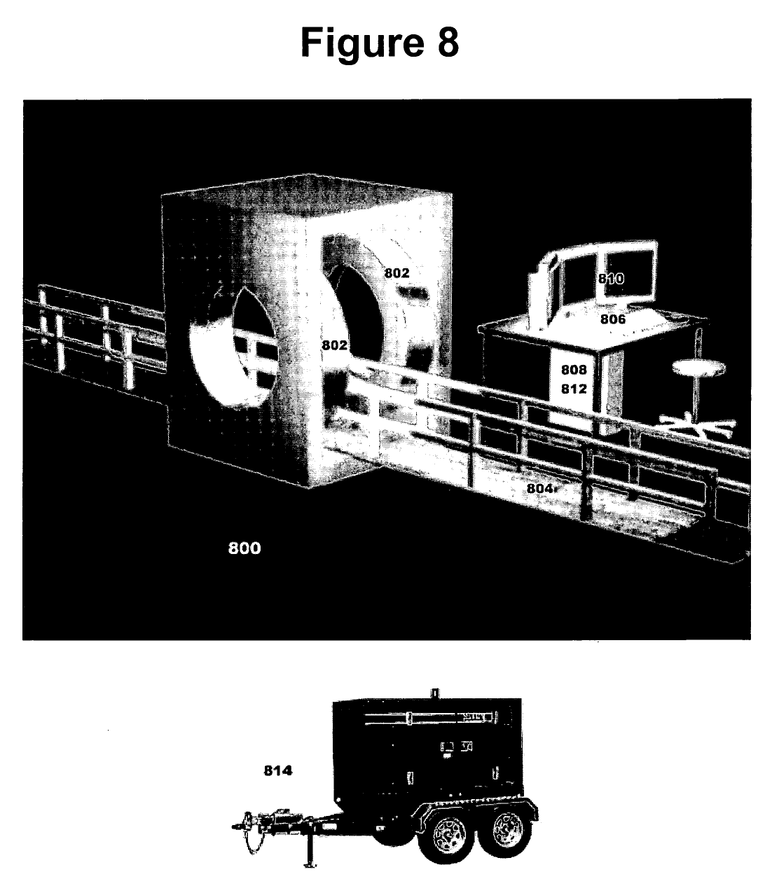

FIG. 8 illustrates an MRI machine of the present invention. MRI machine 800 includes a plurality of magnets 802 disposed opposing each other. The magnets are typically superconducting and generate a field from 1 to 1.5 Tesla; however, fields over 3.0 Tesla can be used. Magnets 802 can be “standard” magnets used in MRI machines or specially designed for light weight or high field strength depending on the image requirements. A passageway 804 acting as a chute for herds of animals is disposed between the magnets to allow the animal to travel between magnets 802. Passageway 804 can be wide enough and magnets 802 spaced far enough apart to accommodate large bulls and deer and elk with full racks of antlers. The width can also accommodate sheep, alone and in a sheep chair. Included in MRI machine 800 is a control station 806 to control the magnets 802 and to capture and analyze the magnetic resonance image. Control station 806 typically has one or more computers 808, monitors 810 and storage devices 812. Further, power generator 814 is included to power the MRI machine 800, by supplying power to the magnets 802 and the control station 806. Sections of the MRI machine can also require lighting; and power generator 814 can supply all ancillary power needs.

A key to a clear image is that the animal is held as still as possible at the time the image is taken. In one embodiment, a handler can walk each animal up the passageway 804 and steady the animal's head between magnets 802 for the time it takes to scan the image. This time can range between 1 and 5 minutes.

Another embodiment includes head gates 818 to restrain the head and neck portion of a cow or bull in a fixed position. Head gates 818 close about a cow's or bull's neck, directly behind and under the head to stabilize it. Head gates 818 can be disposed below passageway 804 and raised once the cow or bull is in position. Alternately, the gates can be disposed along the sides of passageway 804 and can converge on the cow's or bull's neck. Engaging and disengaging head gates 818 can be performed automatically, by a controller at control station 808, or by a handler leading the cow or bull along passageway 804. Head gates 818, in one embodiment, are not made of a material that would leave an artifact on the image. The computer controller can identify the particular machine, date, operator, or have a counter built in to record the number of cows and/or bulls that pass through the machine in a given period.

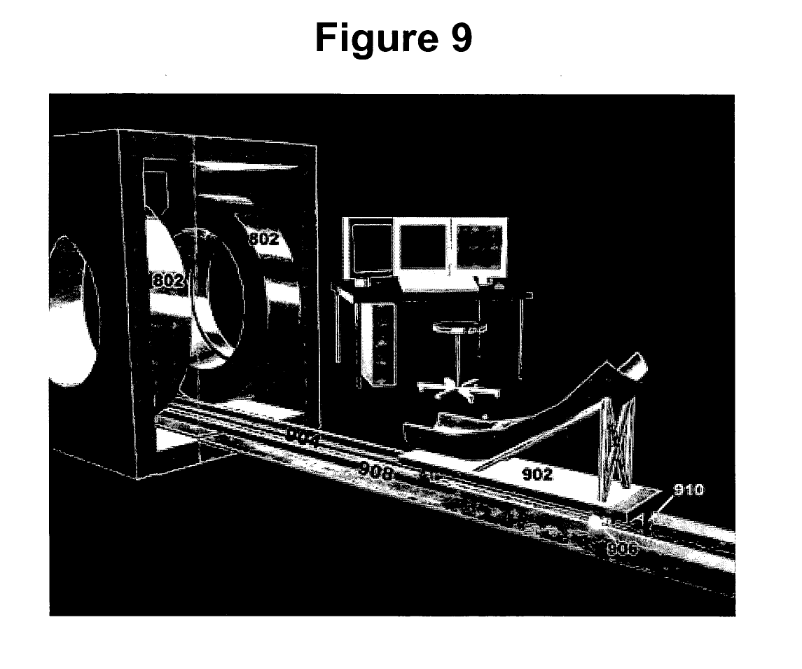

FIG. 9 illustrates an alternate embodiment for sheep. Passageway 904 is designed to receive a sheep chair 902. Sheep chair 902 is a device in which sheep are placed on their backs in a semi-recumbent position. A sheep chair is typically used to handle sheep, and sheep naturally remain still for a few minutes once placed in a chair. Sheep chair 902 can have wheels or skis 906 to allow it to traverse passageway 904 with ease. Further, passageway 904 can include rails 908 to engage wheels 906 to allow for a stable trip down passageway 904.

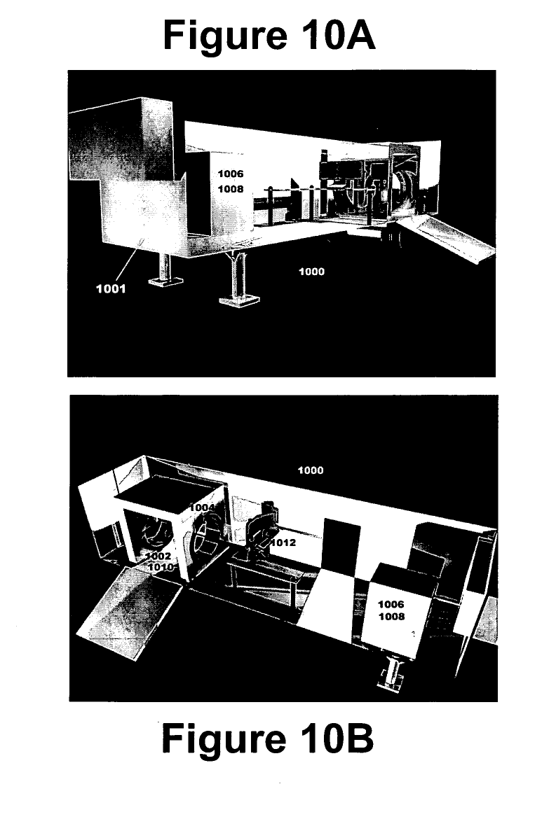

A further embodiment includes mover 910 to automatically propel sheep chair 902 along rails 908 into and out of MRI machine 900. Mover 910 can be a motor mounted to sheep chair 902, a wire guided pulling system, or an engaging mechanism to engage wheels 906 and propel chair 902. FIGS. 10A and 10B illustrate a portable MRI machine 1000. MRI machine 1000 can be mounted on a platform 1001 including a vehicle or a trailer to provide mobility. Typically, MRI machine 1000 and all components can be mounted in a trailer section of a tractor trailer or a rail car. Power can be provided by an external hook up or by, for example, a diesel motor of a cab section of the tractor trailer or can be a separate unit.

MRI machine 1000 is similar to MRI machine 800 in that it includes a plurality of magnets 1002, 1004 disposed opposing each other. The magnets are typically superconducting and generate a field from 1 to 1.5 Tesla; however, fields over 3.0 Tesla can be used. Magnets 1002, 1004 can be “standard” magnets used in MRI machines or specially designed for light weight or high field strength depending on the image requirements.

A passageway 1010 acting as a chute for herds of animals is disposed between the magnets to allow the animal to travel between magnets 1002, 1004. Passageway 1010 can be wide enough and magnets 1002, 1004 spaced far enough apart to accommodate large bulls and deer and elk with full racks of antlers. The width can also accommodate sheep, alone and in a sheep chair.

Included in portable MRI machine 1000 is a control station 1006 to control magnets 1002, 1004 and to capture and analyze the magnetic resonance image. Control station 1006 typically has one or more computers 1008, monitors, and storage devices. Further, power generator 814 is included to power the MRI machine 1000 by supplying power to the magnets 1002, 1004 and the control station 1006. Sections of the MRI machine can also require lighting, and power generator 814 can supply all ancillary power needs. Both the control station 1006 and power generator 814 can be included on platform 1000 or part of a separate platform (not illustrated). Thus, portable MRI machine 1000 can be transported on one or multiple platforms and be brought to the herd to be scanned. The herd does not need to be transported to the MRI machine.

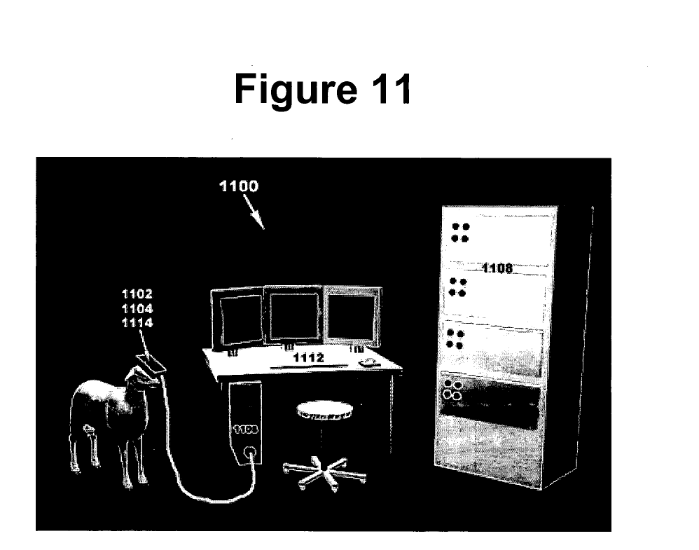

FIG. 11 illustrates a handheld MRI machine 1100. Handheld MRI machine 1100 includes an imager 1102 to place against or around the head of an animal. Magnets 1104 are used to image the animal's head. Handheld MRI 1100 can include a base unit 1106 containing power and a processor to generate and analyze the image. Handheld MRI 1100 and base unit 1106 can be connected during the imaging step.

Alternately, images can be stored in a memory 1108 in the handheld unit 1100 and downloaded to base unit 1106 or transmitted to base unit 1106 over a wireless network, either contemporaneous with the imaging step or after the image has been taken.

A further embodiment includes a reader 1110 to read the distinct markings on the animal's ear tag or brand. The reader can be part of imager 1102 or base unit 1106. This simplifies matching the image to the correct animal. Alternately, handheld MRI 1100 has a keypad 1112, part of imager 1102 or base unit 1106, to allow the operator to manually enter the animal's identification information.

Furthermore, handheld MRI 1100 can process the image with or without displaying the image on the handheld device. Handheld MRI 1100 can have a simple indicator 1114, for example, green, yellow and red indicators to indicate not infected, possible infection or another image is required, and infected.

### EXAMPLES

Sheep: Six control sheep from two different certified scrapie-free flocks were obtained from Pennsylvania. Three deceased Kosher sheep were obtained from a slaughter house in Newark, N.J. One hundred thirteen scrapie sheep, all of a QQ susceptible genotype, were obtained from South Dakota (flock no. SD-1437). The flocks were of mixed breeds, including Hampshire, Suffolk, Brockelface, Western whiteface, and a blackfaced sheep of unknown breed. The sheep ranged from 1-9 years old, with 122 animals total. Two sheep died (scrapie IHC results negative) and were not further tested. Therefore, 120 total sheep were tested. The age of the sheep was important as clinical signs of scrapie often appear in 2-5 year old animals. Clinical signs of scrapie were evaluated by assessing the animals' trembling, hair loss, nibble reflex, and body condition score (a low score indicates a poor body condition).

MRI scanning: MRI scanning was performed on the live sheep and the Kosher sheep. Some sheep were anesthetized and scanned while alive. Most sheep were anesthetized, positioned within the MRI scanner, then euthanized and immediately scanned. No difference was found in the scans of live, anesthetized, or euthanized sheep. Images were acquired on a GE Signa medical scanner at 14×14 cm resolution, 256×160 pixels, at 1 Tesla (except for the Kosher sheep where images were acquired at 1.5 Tesla). T1, T2, proton density, diffusion weighted, FLAIR (Fluid Attenuated Inversion Recovery), and inversion recovery scans were acquired. Slices were 3 mm thick with 0 separation. Sagittal, axial, and dorsal plane images were taken across the brain using standard pre-programmed settings of the MRI. Note, the brain axis is different between humans and sheep.

Image analysis: Representative sagittal sections were either to the left or right of midline. The best section was found to be the section with the lateral ventricle extending into the olfactory bulb. No manipulation was performed on the images. The division by the cerebrum area normalized the data for individual biological variation. Areas were calculated by the use of the Magnetic Lasso Tool within Adobe® Photoshop® and the use of a computer program written to calculate areas using bounding polygons. The Lasso tool settings were: Feather=zero pixels; anti-aliased=on; width=3 pixels; edge contrast=100%; and frequency=100). Both methods of calculation gave identical results.

Immunohistochemistry (IHC) test: Following the MRI exam, each sheep had the immunohistochemistry (IHC) test performed on the tonsils, retropharyngeal nodes, and obex (medulla). Samples were sent to the National Veterinary Services Laboratory in Ames, Iowa testing facility to test for the presence of PrP^{Sc}. Test results are returned as either positive or negative for the presence of PrP^{Sc}.

Third-Eyelid test: The third eyelid live animal test for scrapie was approved by Veterinary Services of the Animal and Plant Health Inspection Service (APHIS) of the United States Department of Agriculture (USDA) as an official test. Samples were sent to the National Veterinary Services Laboratory in Ames, Iowa testing facility to perform tests for the presence of PrP^{Sc}. The results showed either the presence or absence of PrP^{Sc}.

### Example 1

### Correlation Between MRI Image Ratio of Ventricle Area to Cerebrum Area with Scrapie Assessed by Histology

Histogram of Percent Ventricle Area to Cerebrum Area (FIG. 4A)

FIG. 4A shows a histogram generated using the methods of the present invention. Specifically, it shows the number of sheep (totaling 120 tested) plotted against the percent ventricle area of cerebrum area in bins of 0.5%. The sheep were scanned using MRI as described above under “MRI scanning.” The best sagittal section (slice) of the brain was selected (the slice where the lateral ventricle extends into the olfactory lobe); and the area of the lateral ventricle and cerebrum were calculated using the methods listed above in “Image analysis.” The ratio of lateral ventricle area to cerebrum area was calculated by dividing the lateral ventricle area by the cerebrum area. The number of sheep with a particular percent ratio value was separated into bins of 0.005. Each sheep was also tested using the IHC tests as described above in “Immunohistochemistry (IHC) test” and “Third-Eyelid test.” Each sheep was designated as a control which was negative (Control-Neg), as a non-control with scrapie (Scrapie), or as a non-control without scrapie (Negative) as determined by the results of the Ames facility tests.

The histogram shows that there is a point, around the ratio value of 10%-11%, where there is a greater than 84% chance that the sheep tested by the methods of the present invention has scrapie. At a ratio value of 10%, this probability is slightly greater than 84%; at 10.5%, this is 90%; and at 11% and above, this is 100% of the animals tested in this study. Also, there is a sharp decrease in healthy sheep brains and an increase in scrapie-infected brains at this point (thus representing a bimodal distribution). Therefore, there is a distinct point at which a cut-off may be used.

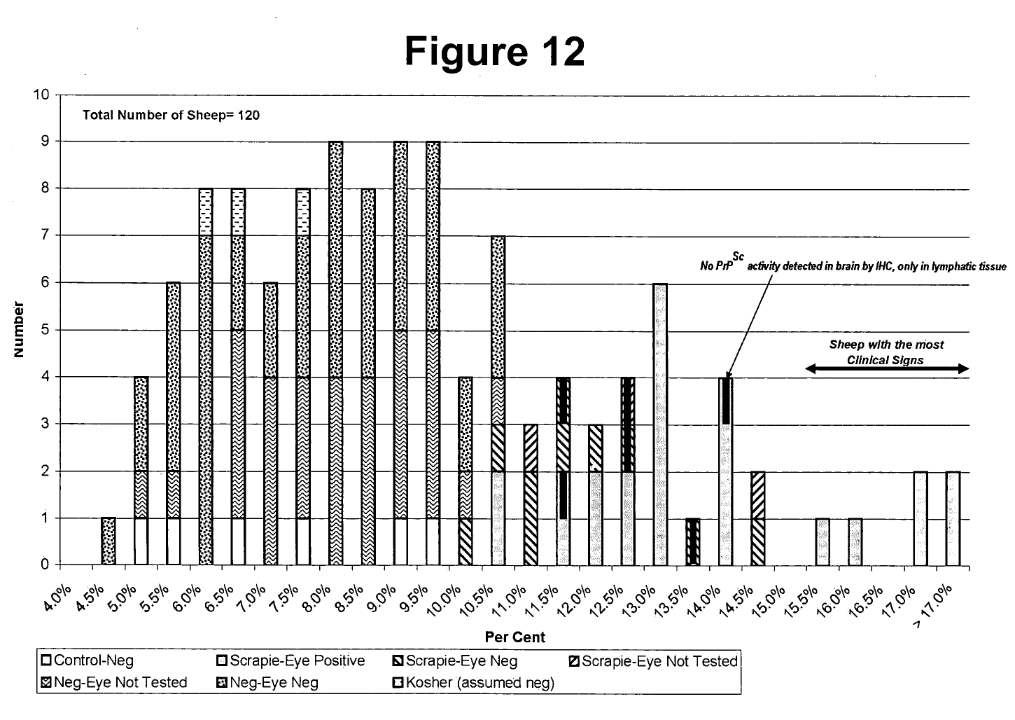

FIG. 12 shows a detailed version of FIG. 4A, showing the break-down of the various test results.

### Example 2

### Correlation Between MRI Image Ratio of Ventricle Area to Cerebrum Area with Scrapie Assessed by Histology

Histogram of (Lateral Ventricle Area)/(cerebrum-lateral ventricle area)*100 (FIG. 4B)

FIG. 4B shows a histogram generated using the methods of the present invention. Specifically, it shows the number of sheep (total 117 tested) plotted against the percent ventricle area of the quantity cerebrum area minus lateral ventricle area in bins of 0.2%. The sheep were scanned using MRI as described above under “MRI scanning.” The best sagittal section (slice) of the brain was selected (the slice where the lateral ventricle extends into the olfactory lobe); and the area of the lateral ventricle and cerebrum were calculated using the methods listed above in “Image analysis.” The ratio was calculated by dividing the lateral ventricle area by the quantity of the cerebrum area minus the lateral ventricle area. This number was then multiplied by 100. The number of sheep with a particular percent ratio value was separated into 0.2% bins. Each sheep was also tested using the IHC tests as described above in “Immunohistochemistry (IHC) test” and “Third-Eyelid test.” Each sheep was designated as a control which was negative (Controls); as a non-control, tested negative by IHC, not tested using the Third-Eyelid test (Not Tested Neg); as a non-control, tested negative for scrapie by both the Third-Eyelid test and also tested negative by IHC (Negative Eyelid); as a non-control, tested negative by Third-Eyelid test, but positive by IHC (NE Positive); as a non-control, not Third-Eyelid tested, but positive IHC (Not Tested Pos); and as a non-control, tested positive using both the Third-Eyelid and IHC tests (Scrapie); as determined by the results of the Ames facility tests.

The histogram shows that there is a point, around a ratio value of 8.2%-9.4%, where there is a greater than 86% chance that the sheep tested by the methods of the present invention has scrapie. At a ratio value of 8.2%, this is greater than 86%; at 8.6%, this is greater than 87%; at 9.0%, this is greater than 90%; and at 9.4% and above, the probability is 96% that a sheep has scrapie, of the animals tested in this study. Also, there is a sharp decrease in healthy sheep brains and an increase in scrapie-infected brains at this point (thus representing a bimodal distribution). Therefore, there is a distinct point at which a cut-off may be used.

In the Example, a binary correlation computer program was run to compare the results of the methods of the present invention to those of using immunohistochemistry. A variable cut-off may be used; and the Example used a cut-off ratio of less than or equal to 8.5% using the lateral ventricle area compared to the cerebrum area. The areas were determined using the bounding polygon area computer program as described above. Anything less than or equal to the cut-off was designated zero (0) for a negative result, and anything greater than the cut-off was designated as a one (1) for a positive result. Using the binary correlation program, a correlation between the results of the MRI methods of the present invention and the results of the IHC test yielded a correlation coefficient of 0.94.

### Example 3

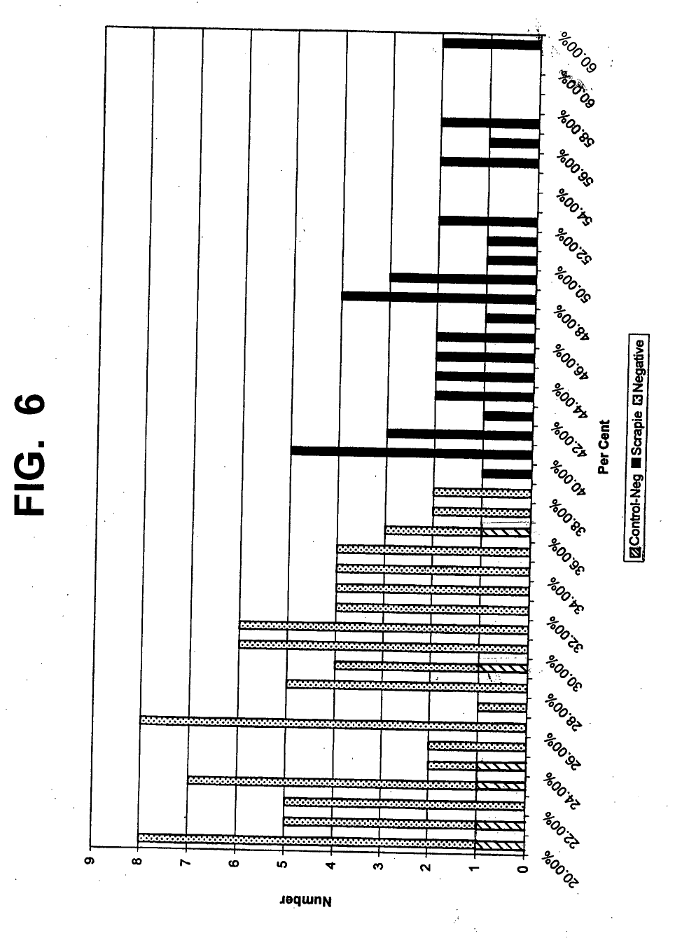

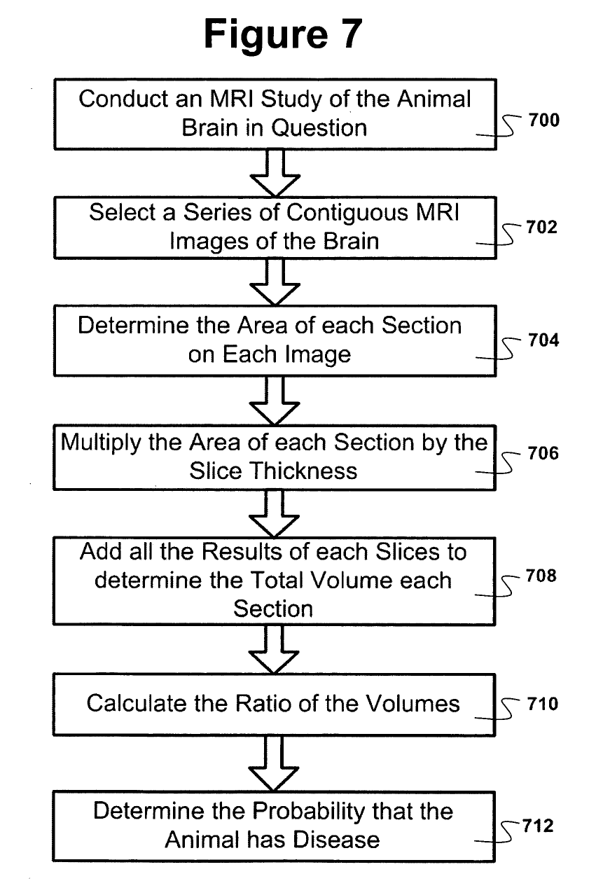

### Histogram of Percent Ventricle Area to Frontal Lobe Area. (FIG. 6)

FIG. 6 shows a histogram generated using the methods of the present invention. Specifically, it shows the number of sheep (totaling 119 tested) plotted against the percent ventricle area of frontal lobe area in bins of 2%. The sheep were scanned using MRI as described above under “MRI scanning.” The best sagittal section (slice) of the brain was selected (the slice where the lateral ventricle extends into the olfactory lobe), and the area of the lateral ventricle and frontal lobe were calculated using the methods listed above in “Image analysis.” The ratio of lateral ventricle area to frontal lobe area was calculated by dividing the lateral ventricle area by the frontal lobe area. The number of sheep with a particular percent ratio value was separated into bins of 2%. Each sheep was also tested using the IHC tests as described above in “Immunohistochemistry (IHC) test” and “Third-Eyelid test.” Each sheep was designated as a control which was negative (Control-Neg), as a non-control with scrapie (Scrapie), or as a non-control without scrapie (Negative) as determined by the results of the Ames facility tests.

The histogram shows that there is a point, around the ratio value of 39%, where there is a 100% chance that the sheep tested has scrapie. Also, there is a sharp decrease in healthy sheep brains and an increase in scrapie-infected brains at this point (thus representing a bimodal distribution). Therefore, there is a distinct point at which a cut-off may be used.

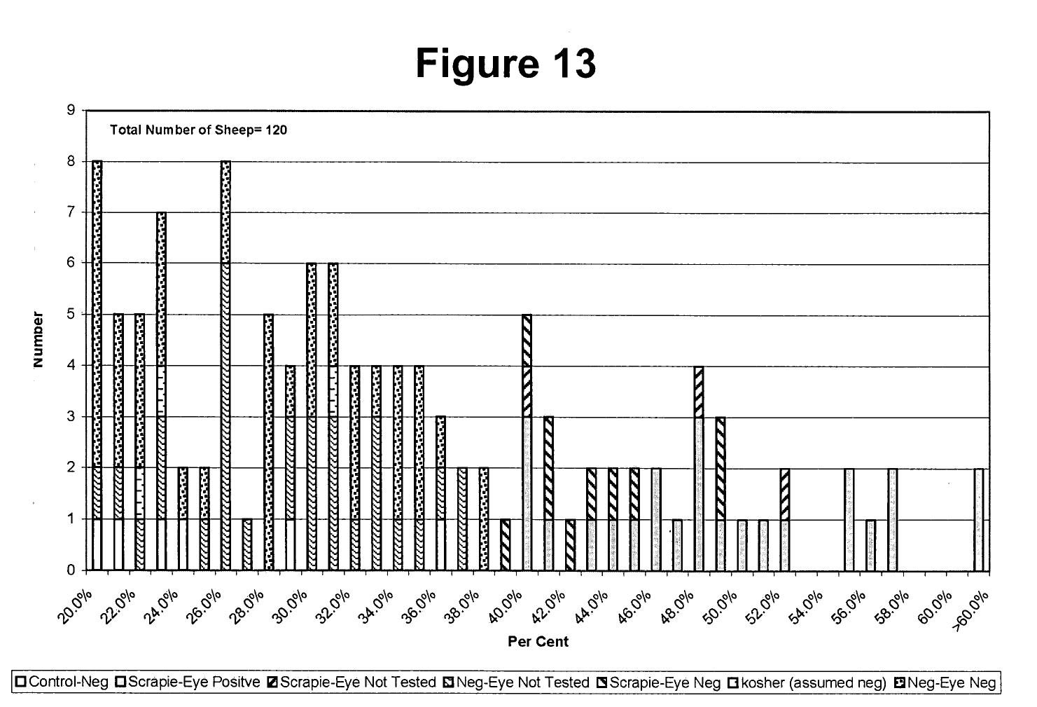

FIG. 13 shows a detailed version of FIG. 6, showing the break-down of the various test results.

### Example 4

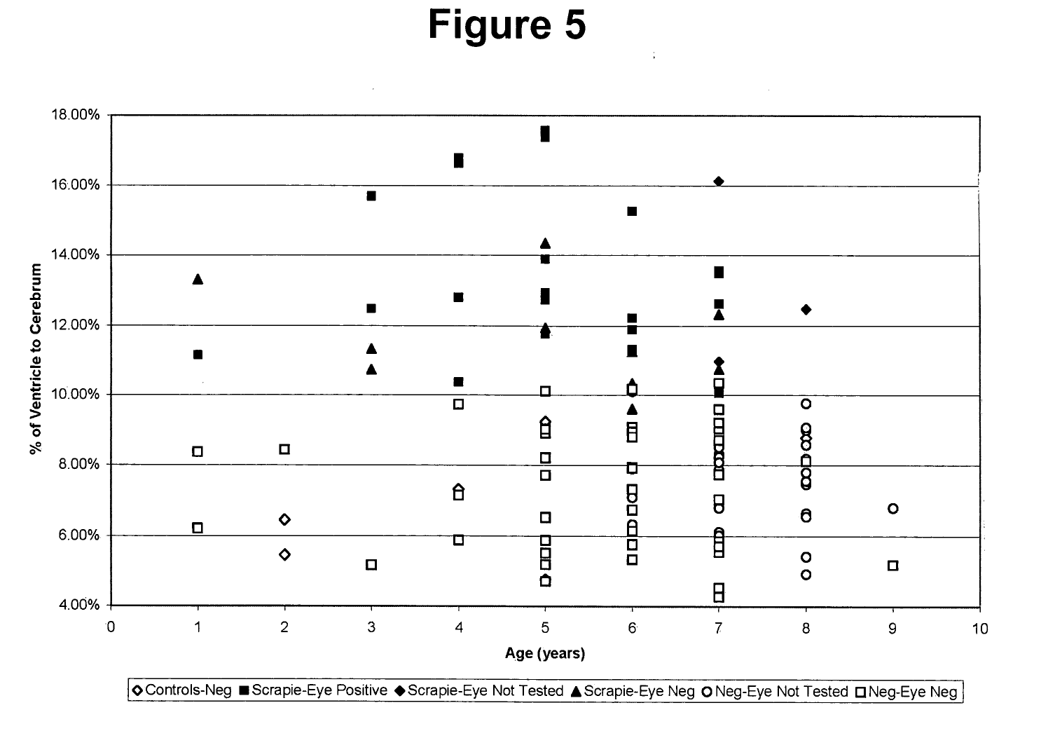

### No Correlation Exists Between Scrapie and the Age of the Tested Sheep. (FIG. 5)

As graphically represented in FIG. 5, plotting the age of the sheep vs. percent ventricle to cerebrum (a method of detecting scrapie in sheep) demonstrates that there is no age correlation between age and detectable disease. Also, changes within the brain of sheep can be observed in sheep as young as one year old.

Further, FIG. 12 illustrates that the present invention can detect TSE prior to detecting PrP^{Sc} in the brain of the animal. Six of the sheep tested were determined to be positive for scrapie from the results of the MRI imaging. However, the pathology determined that PrP^{Sc} was not present in the brains of some of the animals. PrP^{Sc} infection was only found in the lymph nodes. This suggests that PrP^{Sc} presence in the brain is not a requirement for the animal to have physical effects from the disease. Furthermore, most current testing uses the presence of PrP^{Sc} as indication for infection. Thus, the present invention is independent of PrP^{Sc} infection in the brain and can yield more accurate results.

The present invention can be used to compare different structures in the same organ to determine if there are any detectable physical expressions of a disease or infection. The ratio of size of the different structures can be helpful for a physician to diagnose a disease. Further, the method can compare the area or volume of two separate organs to made the same determination.

### Example 5

### Correlation Between MRI Image Ratio of Ventricle Area to Cerebrum Area with Scrapie Assessed by Histology

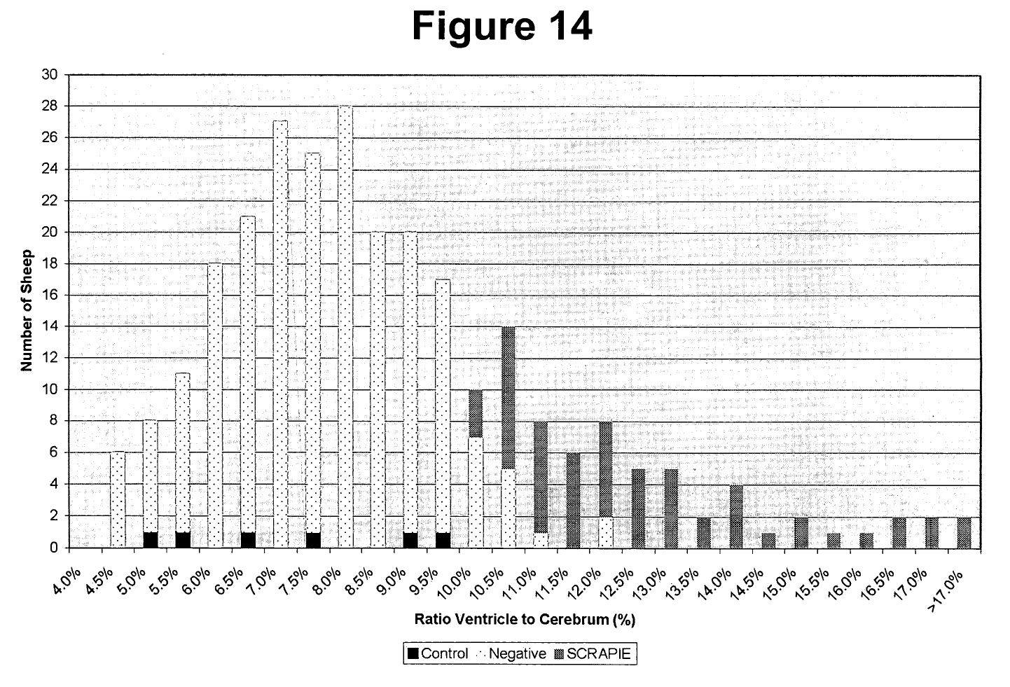

Histogram of Percent Ventricle Area to Cerebrum Area (FIG. 14)

FIG. 14 shows a histogram generated using the methods of the present invention. Specifically, it shows the number of sheep (totaling 274 tested) plotted against the percent ventricle area of cerebrum area in bins of 0.5%. The sheep were scanned using MRI as described above under “MRI scanning.” The best sagittal section (slice) of the brain was selected (the slice where the lateral ventricle extends into the olfactory lobe); and the area of the lateral ventricle and cerebrum were calculated using the methods listed above in “Image analysis.” The ratio of lateral ventricle area to cerebrum area was calculated by dividing the lateral ventricle area by the cerebrum area. The number of sheep with a particular percent ratio value was separated into bins of 0.005. Each sheep was also tested using the IHC tests as described above in “Immunohistochemistry (IHC) test” and “Third-Eyelid test.” Each sheep was designated as a control which was negative (Control-Neg), as a non-control with scrapie (Scrapie), or as a non-control without scrapie (Negative) as determined by the results of the Ames facility tests.

The histogram shows that there is a point, around the ratio value of 10%-12%, where there is a greater than 79% chance that the sheep tested by the methods of the present invention has scrapie. At a ratio value of 10%, this probability is slightly greater than 79%; at 11%, this is greater than 93%; at 11.5%, this is greater than 95%, and at 12.5% and above, this is 100% of the animals tested in this study.

While there have been shown, described, and pointed out fundamental novel features of the invention as applied to a preferred embodiment thereof, it will be understood that various omissions, substitutions, and changes in the form and details of the devices illustrated, and in their operation, may be made by those skilled in the art without departing from the spirit and scope of the invention. For example, it is expressly intended that all combinations of those elements and/or steps which perform substantially the same function, in substantially the same way, to achieve the same results are within the scope of the invention. Substitutions of elements from one described embodiment to another are also fully intended and contemplated. It is also to be understood that the drawings are not necessarily drawn to scale, but that they are merely conceptual in nature.

What is claimed is:

1. A method for detecting the presence of a transmissible spongiform encephalopathy (TSE) in a mammal comprising the steps of:
(a) taking a magnetic resonance imaging (MRI) section of the brain of the mammal;
(b) measuring a first brain structure in the section;
(c) measuring a second brain structure in the section;
(d) calculating the ratio of the measurements of the first brain structure to the second brain structure; and
(e) using the ratio to determine the probability that the mammal has a TSE.

2. A method for detecting the presence of a transmissible spongiform encephalopathy (TSE) in a mammal comprising the steps of:
(a) taking a magnetic resonance imaging (MRI) image of the brain of the mammal;
(b) selecting multiple sections within the image;
(c) determining an area of each section;
(d) multiplying the areas by a thickness of each section;
(e) adding the selected sections together to determine a volume of the image;
(f) measuring a volume of a first brain structure in the section;
(g) measuring a volume of a second brain structure in the section;
(h) calculating the ratio of the volumes of the first brain structure to the second brain structure; and
(i) using the ratio to determine the probability that the mammal has a TSE.

3. The method of claim 1, the measuring steps measure an area of the first brain structure and the second brain structure.

4. The method of claim 1 wherein the measuring steps measure a volume.

5. The method of claim 1 wherein the mammal is living.

6. The method of claim 5 wherein the mammal is one of sedated and anesthetized.

7. The method of claim 1 wherein the mammal is one of ovine, bovine, and cervid.

8. The method of claim 1 wherein the section is at least one of a sagittal and an axial section of the brain.

9. The method of claim 1 wherein the first brain structure is at least one of a lateral ventricle and a frontal lobe.

10. The method of claim 1 wherein the second brain structure is a cerebrum.

11. The method of claim 1 wherein the measuring steps taken include using a bounding polygon tool.

12. The method of claim 1 wherein the TSE is selected from the group consisting of scrapie, bovine spongiform encephalopathy (BSE), chronic wasting disease (CWD), transmissible mink encephalopathy (TME), feline spongiform encephalopathy (FSE), and Creutzfeldt-Jakob disease (CJD).

13. The method of claim 1 wherein a portable MRI is used to take the image.

14. The method of claim 13 wherein the portable MRI has a brace to hold the head of a living mammal.

15. The method of claim 13 wherein the portable MRI has a convex holding area to hold a living mammal.

16. The method of claim 1 wherein using the ratio to determine the probability that the mammal has a TSE is methodology dependent wherein ratio values will change using different brain structures.

17. The method of claim 1 wherein parameter settings for the MRI are a Fast Spin Echo Sequence, a TE (echo time) equal to 180 milliseconds, a TR (repetition time) equal to 4000 milliseconds, a FOV (field of view) equal to 14 centimeters, slice thickness=3 millimeters (contiguous).
